(19) United States
(12) Patent Application Publication
(10) Pub. No.: US 2025/0082881 A1
(43) Pub. Date: Mar. 13, 2025

(54) FLUID RESPONSIVENESS MONITORING

(71) Applicant: KONINKLIJKE PHILIPS N.V., EINDHOVEN (NL)

(72) Inventors: PATRICK SCHONLEITNER, EINDHOVEN (NL); ESTHER MARJAN VAN DER HEIDE, 's-Hertogenbosch (NL); FEI ZUO, EINDHOVEN (NL); MEYKE ROOSINK, EINDHOVEN (NL); JEFFREY VISSER, EINDHOVEN (NL)

(21) Appl. No.: 18/882,377

(22) Filed: Sep. 11, 2024

Related U.S. Application Data
(60) Provisional application No. 63/537,573, filed on Sep. 11, 2023.

(51) Int. Cl. A61M 16/00; G16H 10/60; G16H 20/40

(52) U.S. Cl. CPC A61M 16/024; A61M 16/0066; G16H 10/60; G16H 20/40; A61M 2205/502; A61M 2205/583

(57) ABSTRACT

A system (100) includes a controller (130) with a memory (131) that stores instructions, and a processor (132) that executes the instructions. When executed by the processor (132), the instructions cause the system (100) to: automatically gather patient information of a patient; rank clinically validated tests for fluid responsiveness based on the automatically gathered patient information; identify a clinically validated test ranked highest; repeatedly perform the clinically validated test ranked highest to monitor fluid responsiveness of the patient; determine whether to continue performing the clinically validated test ranked highest; and output guidance for performing an alternative clinically validated test based on determining not to continue performing the clinically validated test ranked highest.

### BACKGROUND

Hemodynamics are the dynamics of blood flow. Fluid therapy is a cornerstone of hemodynamic management in critically ill patients. Perfusion is the passage of a fluid through a natural channel or tissue. The goal of fluid therapy is to maintain organ perfusion by increasing cardiac output (CO) via fluid loading to expand circulating blood volume. However, in a subset of patients, additional fluid loading does not further improve cardiac output. Administering fluid beyond a patient specific threshold can lead to no improvement of cardiac output and clinically relevant fluid overload and higher morbidity and mortality. To avoid such detrimental effects, it is crucial to continuously monitor fluid responsiveness for some patients. Currently there is no standardized method for a continuous structured approach in clinical practice to assess if a patient will benefit from fluid application. The absence of such a continuous structured approach is relevant for determining fluid responsiveness in patients including critically ill patients before the application of fluids. Several tests and protocols have been clinically validated but rely on extensive user input and/or require specific patient conditions. As a result, fluid responsiveness testing is currently carried out infrequently and in the form of spot checks with multiple steps that are time intensive and difficult to record/capture. Furthermore, the choice of fluid responsiveness test is currently often not tailored to the patient's condition. Tests may be carried out in patients without fulfilling all test requirements such as Tidal volume or lung compliance of a given test, resulting in false negative assessments and false positive test assessments.

### SUMMARY

According to an aspect of the present disclosure, a system includes a controller with a memory that stores instructions, and a processor that executes the instructions. When executed by the processor, the instructions cause the system to: automatically gather patient information of a patient; rank clinically validated tests for fluid responsiveness based on the automatically gathered patient information; identify a clinically validated test ranked highest; repeatedly perform the clinically validated test ranked highest to monitor fluid responsiveness of the patient; determine whether to continue performing the clinically validated test ranked highest; and output guidance for performing an alternative clinically validated test based on determining not to continue performing the clinically validated test ranked highest.

According to another aspect of the present disclosure, a method for monitoring fluid responsiveness includes automatically gathering patient information of a patient; ranking clinically validated tests for fluid responsiveness based on the automatically gathered patient information; identifying a clinically validated test ranked highest; repeatedly performing the clinically validated test ranked highest to monitor fluid responsiveness of the patient; determining whether to continue performing the clinically validated test ranked highest; and outputting guidance for performing an alternative clinically validated test based on determining not to continue performing the clinically validated test ranked highest.

According to another aspect of the present disclosure, a tangible, non-transitory computer-readable medium stores instructions. When executed by a processor, the instructions cause the processor to: automatically gather patient information of a patient; rank clinically validated tests for fluid responsiveness based on the automatically gathered patient information; identify a clinically validated test ranked highest; repeatedly perform the clinically validated test ranked highest to monitor fluid responsiveness of the patient; determine whether to continue performing the clinically validated test ranked highest; and output guidance for performing an alternative clinically validated test based on determining not to continue performing the clinically validated test ranked highest.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 illustrates a system for fluid responsiveness monitoring, in accordance with a representative embodiment.

FIG. 2 illustrates a hybrid flowchart for fluid responsiveness monitoring, in accordance with a representative embodiment.

FIG. 3 illustrates a method for fluid responsiveness monitoring, in accordance with a representative embodiment.

FIG. 4 illustrates a computer system, on which a method for fluid responsiveness monitoring is implemented, in accordance with another representative embodiment.

### DETAILED DESCRIPTION

In the following detailed description, for the purposes of explanation and not limitation, representative embodiments disclosing specific details are set forth in order to provide a thorough understanding of embodiments according to the present teachings. However, other embodiments consistent with the present disclosure that depart from specific details disclosed herein remain within the scope of the appended claims. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. Definitions and explanations for terms herein are in addition to the technical and scientific meanings of the terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below.

As described herein, a fluid responsiveness monitoring system and method can rank clinically validated tests/procedures of fluid responsiveness based on automatically gathered patient information. The fluid responsiveness monitoring system and method can use the most preferred test and whenever possible conduct the test continuously and autonomously or when necessary, and guide the medical personnel through alternative tests that require medical personnel intervention. Thereby the fluid responsiveness monitoring system and method can support the medical personnel to optimize fluid therapy in critically ill patients by providing continuous reliable information of a patients' fluid responsiveness, ultimately improving patient outcomes.

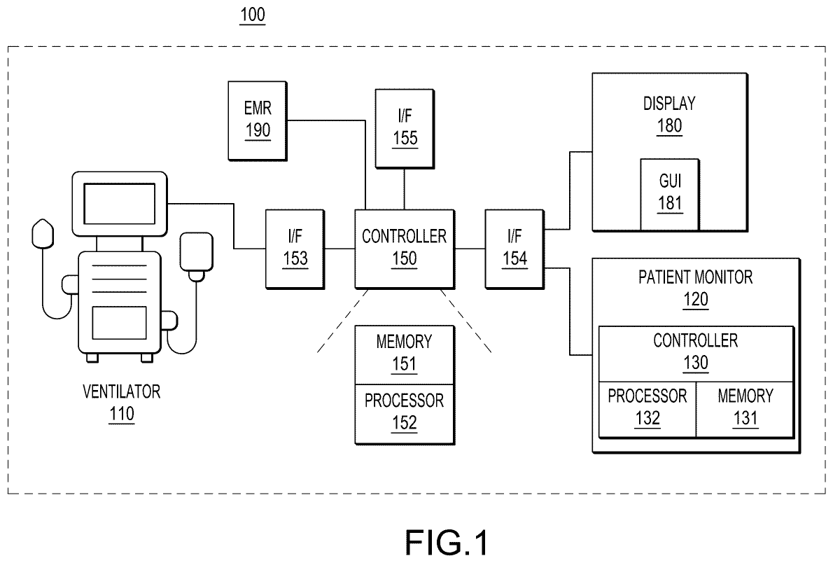

FIG. 1 illustrates a system 100 for fluid responsiveness monitoring, in accordance with a representative embodiment.

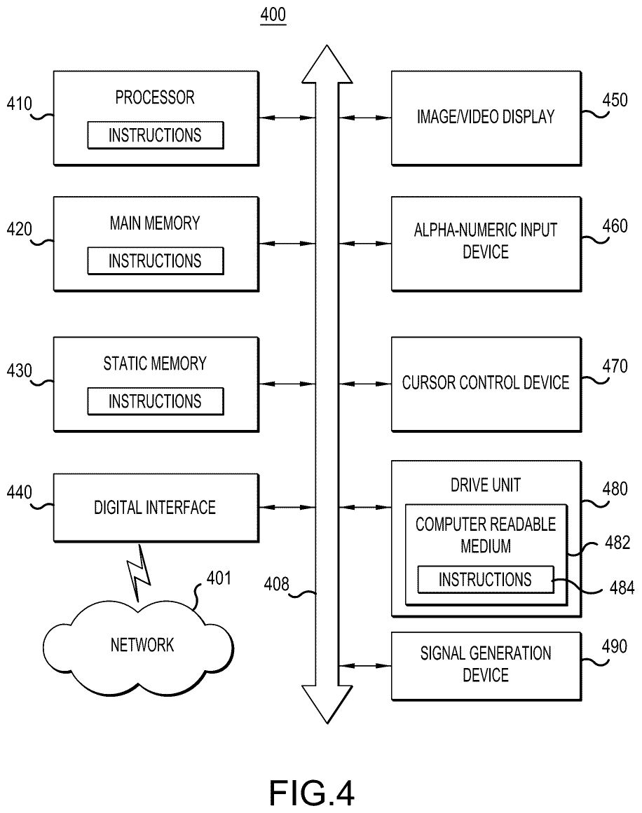

The system 100 in FIG. 1 is a system for fluid responsiveness monitoring and includes components that may be provided together or that may be distributed. The system 100 includes a ventilator 110, a patient monitor 120, a controller 150, a display 180, and an EMR 190 (electronic medical records system). The patient monitor 120 includes a controller 130, and the controller 130 includes a memory 131 that stores instructions and a processor 132 that executes the instructions. The controller 150 includes a memory a memory 151 that stores instructions, a processor 152 that executes the instructions, a first interface 153, a second interface 154, and a third interface 155. A computer that can be used to implement the controller 150 is depicted in FIG. 4, though a controller 150 may include more elements than depicted in FIG. 1 and more or fewer elements than depicted in FIG. 4. In FIG. 1, the controller 150 may be a first controller, the memory 151 may be a first memory, the processor 152 may be a first processor, the controller 130 may be a second controller, the memory 131 may be a second memory, and the processor 132 may be a second processor.

The ventilator 110 is used to provide artificial ventilation to a patient. The ventilator 110 is connected to the controller 150 via the first interface 153. The patient monitor 120 and the display 180 are connected to the controller 150 via the second interface 154. The third interface 155 may comprise a user interface.

The patient monitor 120 may be used to receive patient information such as vital sign readings from one or more connected peripheral devices, and display the patient information such as vital sign readings on a display. As such, the patient monitor 120 may include dedicated interfaces that connect the patient monitor 120 to connected peripheral devices, and a screen configured to display patient information as text, waveforms, icons, or other types of displayable information.

The display 180 may be local to the controller 150 or may be remotely connected to the controller 150. The second interface 154 may connect the display 180 to the controller 150 as a local wired interface such as an Ethernet cable or as a local wireless interface such as a Wi-Fi connection. The display 180 may be interfaced with other user input devices by which medical personnel can input instructions, including mouses, keyboards, thumbwheels and so on. The display 180 may be a monitor such as a computer monitor, a display on a mobile device, an augmented reality display, a television, an electronic whiteboard, or another screen configured to display electronic imagery. The display 180 may also include one or more input interface(s) such as those noted above that may connect to other elements or components, as well as an interactive touch screen configured to display prompts to medical personnel and collect touch input from medical personnel. The controller 150 may execute instructions to cause the system 100 to display a result of monitoring fluid responsiveness on the display 180.

The EMR 190 may be provided for a facility that includes the system 100, or for multiple facilities including the facility that provides the system 100. The EMR 190 may be configured to provide information on patient conditions, patient statuses, patient histories, and other information that may be relevant to the fluid responsiveness monitoring described herein.

The ventilator 110, the patient monitor 120, the controller 150, and the display 180 may include additional interfaces. One or more of the interfaces shown or not shown may include ports, disk drives, wireless antennas, or other types of receiver circuitry that connect the ventilator 110, the patient monitor 120, the controller 150, the display 180 and/or other electronic elements. One or more of the interfaces may also include user interfaces such as buttons, keys, a mouse, a microphone, a speaker, a display separate from the display 180, or other elements that medical personnel can use to interact with the ventilator 110, the patient monitor 120, the controller 150 and/or the display 180 such as to enter instructions and receive output.

The controller 150 may perform some of the operations described herein directly and may implement other operations described herein indirectly. For example, the controller 150 may indirectly control operations such as by generating and transmitting content to be displayed on the display 180. The controller 150 may directly control other operations such as logical operations performed by the processor 152 executing instructions from the memory 151 based on input received from electronic elements and/or medical personnel via the interfaces. Accordingly, the processes implemented by the controller 150 when the processor 152 executes instructions from the memory 151 may include steps not directly performed by the controller 150.

As set forth above, the system 100 includes a controller 150 with a memory 151 that stores instructions, and a processor 152 that executes the instructions. When executed by the processor, the instructions executed by the processor 152 cause the system 100 to: automatically gather patient information of a patient; rank clinically validated tests for fluid responsiveness based on the automatically gathered patient information; identify a clinically validated test ranked highest; repeatedly perform the clinically validated test ranked highest to monitor fluid responsiveness of the patient; determine whether to continue performing the clinically validated test ranked highest; and output guidance for performing (an alternative) clinically validated test based on determining not to continue performing the clinically validated test ranked highest. In some embodiments, the system 100 may autonomously detect the completion of a test step via data from the patient monitor 120 or data from the ventilator 110 or data from a camera-based system to detect the steps taken by the caretaker.

The repeated test ranking performed by the system 100 may be performed continuously or semi-continuously. The repeated test ranking may be performed at pre-determined intervals or at intervals dynamically set for the patient or otherwise by medical personnel. The timing of the repeated test ranking may also be adjusted such as dynamically based on triggers for the patient reading when a patient reading exceeds a threshold.

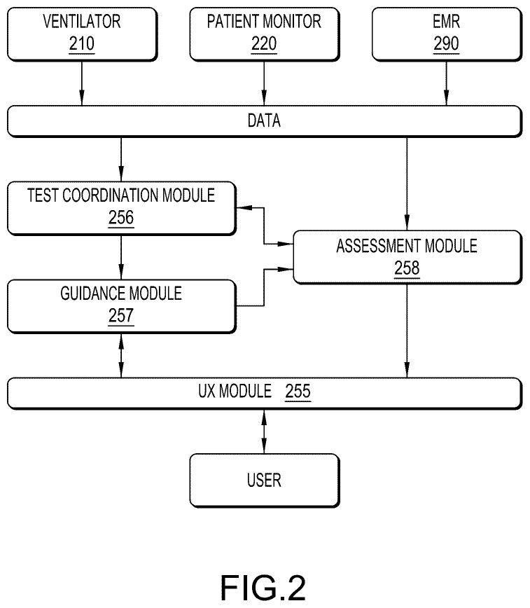

FIG. 2 illustrates a hybrid flowchart for fluid responsiveness monitoring, in accordance with a representative embodiment.

The hybrid flowchart of FIG. 2 may be performed by the system 100 including the controller 150 based on input data from the ventilator 110 and the patient monitor 120, as well as input data from an electronic medical records system.

The hybrid flowchart in FIG. 2 includes inputting data from a ventilator 210, a patient monitor 220, and an EMR 290 (electronic medical records system). The ventilator 210 comprises a mechanical ventilator. The input data may be received by a controller such as the controller 150 in FIG. 1. The controller may implement a test coordination module 256, a guidance module 257 and an assessment module 258, such as by software instructions stored in the memory 151 and executed by the processor 152 in the controller 150 in FIG. 1. The patient monitor 120 or the display 180 may implement a UX module 255, such as by software instructions stored in the memory 131 and executed by the processor 132 in the controller 130 in FIG. 1.

The controller may obtain as input ventilator data from the ventilator 210, patient monitor data from the patient monitor 220, and EMR data from the EMR 290. The input data from the ventilator 210, the patient monitor 220 and the EMR 290 in FIG. 2 is provided to the test coordination module 256 and to the assessment module 258. The controller 150 in FIG. 1 may automatically gather or otherwise receive patient information from the input data to provide to the test coordination module 256 and to the assessment module 258.

The test coordination module 256 evaluates the input data and other types of information to rank one or more clinically validated test(s) and identify a clinically validated test ranked highest. The ranking ranks the clinically validated tests for fluid responsiveness based on the automatically gathered patient information, and may reflect specific characteristic data of the patient. The test coordination module 256 provides the highest ranked clinically validated test to the assessment module 258 initially for confirmation and then, once confirmed, to the guidance module 257. The guidance module 257 provides guidance for the highest ranked clinically validated test to the UX module 255 once the highest ranked clinically validated test is confirmed. The assessment module 258 assesses the highest ranked clinically validated test from the test coordination module 256 in view of the input data from the ventilator 210, the patient monitor 220 and the EMR 290, to determine whether any input or other type of interaction is required. The assessment module 258 may confirm the highest ranked clinically validated test, or otherwise indicate back to the test coordination module 256 whether input or other type of interaction is inadvisable so that the test coordination module 256 should re-rank the highest ranked clinically validated test. In some embodiments, feedback from the assessment module 258 may specifically exclude the highest ranked clinically validated test based on the types of interaction required from the patient or a medical professional. Once confirmed, the assessment module 258 also provides indications of the types of interaction required for the highest ranked clinically validated test to the UX module 255.

The test coordination module 256 and/or the assessment module 258 may also provide the rankings of multiple or even all clinically validated tests assessed by the test coordination module 256 to the UX module 255, to show the clinically validated test ranked highest in the context of other clinically validated tests not ranked highest. In some embodiments, the clinically validated test ranked highest may be overruled by clinical staff, such that showing the context of the other clinically validated test(s) not ranked highest may be referenced by the clinical staff to determine when to overrule the clinically validated test ranked highest.

The guidance module 257 provides guidance for the clinically validated test ranked highest for output to the UX module 255 (user interface). The guidance module 257 may use an algorithm or a table to determine which types of interaction are required for each type of clinically validated test. The guidance provided by the guidance module 257 is not limited to guidance for a new clinically validated test to be used when changing between clinically validated tests. Additionally, not every change between clinically validated tests requires guidance. Rather, guidance may be provided for how to do perform certain clinically validated tests, such as a manual test that requires user input. Guidance may also be provided for an existing clinically validated test that already needs input from the user. A change between clinically validated tests may be appropriate for a variety of circumstances, such as when an automated test currently being used is based on only waveform and EMR data but is no longer suitable.

The UX module 255 interacts with the medical personnel as a user interface, and may provide output for a display, a speaker, for the patient monitor 220, for the display 180 in FIG. 1, or as another type of output perceptible to medical personnel.

The assessments and rankings from the hybrid flowchart in FIG. 2 may be repeated. The controller 150 may repeatedly perform the clinically validated test ranked highest to monitor fluid responsiveness of the patient and determine whether to continue performing the clinically validated test ranked highest based on new input data from the ventilator 210, the patient monitor 220 and/or the EMR 290. When the controller 150 determines that a new clinically validated test should be performed next, the controller 150 may output guidance from the guidance module 257 for performing an alternative clinically validated test based on the assessment module 258 assessing that the current highest ranked clinically validated test should no longer be used. The switch from one clinically validated test to another may be for changed rankings and/or for another reason. In some embodiments, the controller 150 may autonomously detect the completion of a test step via data from the patient monitor 280 or data from the ventilator 210 or from the EMR 290 or data from a camera-based system to detect the steps taken by the caretaker.

The test coordination module 256, the guidance module 257, and the assessment module 258 may comprise software stored in the memory 151 and executed by the processor 152, and/or circuits implemented by circuitry such as application-specific integrated circuits (ASICs), field-programmable gate arrays (FPGAs), or other types of circuits. The test coordination module 256, the guidance module 257, and the assessment module 258 are in more detail described next.

The test coordination module 256 uses input from the patient monitor 220 such as vital sign data, input from the ventilator 210, and available patient records from the EMR 290 to rank different fluid responsiveness tests based on the current patient condition. The test coordination module 256 may advise on the selection of the most suitable fluid responsiveness test. In more detail, the test coordination module 256 performs two or more steps. As a first step, the test coordination module 256 determines the eligibility of available fluid responsiveness tests by comparing the prerequisites for the available fluid responsiveness tests with the patient condition(s) by collecting or otherwise obtaining information from the patient monitor 280, the ventilator 210 and the EMR 290. The collected information may include, for example, breathing status, presence of specific conditions, Tidal volume, and the primary diagnosis and comorbidities. The breathing status may reflect whether breathing is spontaneous or ventilated. The specific conditions may include the presence of arrythmias, intraabdominal hypertension, thorax instabilities or other types of conditions. The Tidal volume may be checked for lung compliance. As a second step, the test coordination module 256 may rank each eligible test based on additional information. The additional information may include performance of the test, need/urgency to apply the test, degree of invasiveness, and/or applicability. Performance of the test may reflect known reliability of the test such as sensitivity/specificity, given the current patient condition. Context information and previous test results may be used as input here to indicate performance of the test. Need/urgency to apply the test may vary for different groups insofar as certain patient groups/conditions it is desirable to use a highly accurate test or to apply the test more frequently, such as to avoid high risks associated with fluid overload. Applicability may reflect whether the test can be used without additional procedures, or if the test can be directly derived from existing waveform data without medical personnel intervention needed. Based on the above information, the test coordination module 256 derives a final test suitability score for each test. The final test suitability may be a binary result or may have a wider range such as a number ranging from 0 to 100, to indicate the most desirable test. The derivation of the final test suitability score may be based on a weighted average of all factors listed above. The weights may be determined manually or derived from an existing data set where the optimal test selection is known together with the other relevant information. In the case where weights are derived from an existing data set, a regression model may be used to derive the optimal weights. The test coordination module 256 provides the highest ranked clinically validated test to the assessment module 258 initially for confirmation, and to the guidance module 257 once confirmed.

The assessment module 258 collects and analyses available ventilator waveform and hemodynamic waveform data to evaluate fluid responsiveness. The assessment module 258 may assess the certainty of a fluid responsiveness prediction and either display the result or prompt additional fluid responsiveness testing in case of high uncertainty. When the selected fluid responsiveness test can be performed without additional medical personnel input or intervention the assessment module 258 may be triggered directly. Otherwise, the assessment module 258 may trigger the guidance module 257 to provide interactive guidance to the medical personnel for performing the selected fluid responsiveness test. Additionally, the test coordination module 256 may supply the assessment module 258 with patient condition-based limits for the selected test. The assessment module 258 may be triggered by the test coordination module 256 or by the guidance module 257. In case the patient condition permits fluid responsiveness determination from available waveform data the assessment module 258 will continuously estimate a patients fluid responsiveness by calculating, for example, the pulse pressure or stroke volume changes over a respiratory cycle. When the test result is outside prespecified limits with respect to the patient's condition the test coordination module 256 will reevaluate the optimal testing procedure based on feedback from the assessment module 258. If the test coordination module 256 determines that an automated test of fluid responsiveness should be performed the assessment module 258 can trigger the test at a set interval until the condition changes or the test value falls outside the prespecified limits in which case the test coordination module 256 can reevaluate the optimal testing procedure. Once a patient's fluid responsiveness has been determined, the assessment module 258 may supply the UX module 255 with information regarding the test results.

The guidance module 257 may support the medical personnel by providing step-by-step instructions of the selected fluid responsiveness test, record start and end time and related patient data. The guidance module 257 may be triggered by the test coordination module 256 when the medical personnel needs to perform an intervention to determine a person's fluid responsiveness. Based on the test suggested by the test coordination module 256 the guidance module 257 may provide step-by-step instructions via the UX module 255. After each step, the guidance module 257 may require the medical personnel to acknowledge the completion of a protocolized test step until all steps are completed. The guidance module 257 may record time stamps for the test steps such as start and end of a test step, and supply the assessment module 258 with the required timing information to evaluate fluid responsiveness. In some embodiments, the guidance module 257 may inform the medical personnel via voice command.

The UX module 255 may include several submodules such as windows or screens for 1.) displaying a patient fluid responsiveness, 2.) guiding the medical personnel through manual test steps, and 3.) altering protocols and settings. That is, the UX module 255 may include at least an indicator window, a guidance window, and a protocol and setting window as sub-modules. The indicator window may receive information from the assessment module 258 and display the result of fluid responsiveness testing in the form of a numerical value, or any other UX element representing a continuous scale or binary state. The output of the result of fluid responsiveness testing may be in the form of a latest numerical value, the latest numerical values, or a trend view with added data. Data added for a trend view may include times when tests were performed resulting in the latest numerical values, details of the specific tests, and details of any interventions performed. The guidance window may display text, animation, pictures, and videos guiding the medical personnel through tests of fluid responsiveness in a comprehensive step-by-step approach. The guidance window may display UX elements allowing the medical personnel to acknowledge the completion of test step. The protocol and setting window may be used by medical personnel to change tests, test steps, desirability-of-test settings, test limits or trigger manual tests.

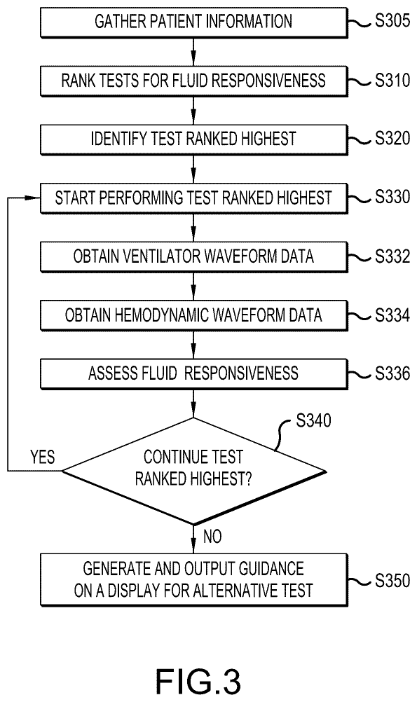

FIG. 3 illustrates a method for fluid responsiveness monitoring, in accordance with a representative embodiment.

At S305, the method of FIG. 3 starts with gathering patient information. The patient information may be received or otherwise gathered by the controller 150.

At S310, tests are ranked for fluid responsiveness. The tests may be ranked by the test coordination module 256 implemented by the controller 150.

At S320, a test ranked highest may be identified. The highest ranked test may be identified by the test coordination module 256 implemented by the controller 150. Although not shown, the highest ranked test may be provided for assessment to the assessment module 258.

At S330, performance of the test ranked highest may be started. The assessment module 258 may initiate the highest ranked test, and assess feedback from the ventilator 250, the patient monitor 220 and the EMR 290.

At S332, ventilator waveform data is obtained. The ventilator waveform data is obtained from the ventilator 210 by the assessment module 258 as a form of feedback for the current test being administered.

At S334, hemodynamic waveform data is obtained. The hemodynamic waveform data may be obtained from the patient monitor 220 by the assessment module 258 as a form of feedback for the current test being administered. Other types of data from peripheral devices connected to the patient monitor 220 may also be obtained at S334.

At S336, fluid responsiveness is assessed. The fluid responsiveness is assessed by the assessment module 258 based on the data received from the ventilator 210, the patient monitor 220 and the EMR 290, and in view of the current test being administered.

At S340, a determination is made as to whether to continue the test ranked highest based on assessing fluid responsiveness of the patient. The determination at S340 may be performed by the assessment module 258, and may be performed continuously as tests are administered to the patient. A determination to continue or not continue may be based on the data received from the ventilator 210, the patient monitor 220 and/or the EMR 290. In some embodiments, the determination at S340 may be based on additional information from other peripheral devices or even interactive input from medical personnel.

If the test ranked highest is to be continued (S340=Yes), the method returns to S330. When the fluid responsiveness of the patient is assessed to be uncertain, the controller 150 may output the guidance for performing an alternative clinically validated test based on determining not to continue performing the clinically validated test ranked highest. If the test ranked highest is not to be continued (S340=No), at S350 the method of FIG. 3 includes generating and outputting guidance on a display for an alternative test.

As noted earlier with respect to FIG. 2, the guidance provided in a method such as in FIG. 3 is not limited to guidance for a new clinically validated test to be used when changing between clinically validated tests. Additionally, not every change between clinically validated tests requires guidance. Rather, guidance may be provided for how to do perform certain clinically validated tests, such as a manual test that requires user input. A change between clinically validated tests may be appropriate for a variety of circumstances, such as when an automated test currently being used is based on only waveform and EMR data but is no longer suitable. In some embodiments, the original highest ranked test may require guidance that is provided periodically, such as each time the determination at S340 is performed and results in a determination that the test ranked highest is to be performed.

FIG. 4 illustrates a computer system, on which a method for fluid responsiveness monitoring is implemented, in accordance with another representative embodiment.

Referring to FIG. 4, the computer system 400 includes a set of software instructions that can be executed to cause the computer system 400 to perform any of the methods or computer-based functions disclosed herein. The computer system 400 may operate as a standalone device or may be connected, for example, using a network 401, to other computer systems or peripheral devices. In embodiments, a computer system 400 performs logical processing based on digital signals received via an analog-to-digital converter.

In a networked deployment, the computer system 400 operates in the capacity of a server or as a client user computer in a server-client user network environment, or as a peer computer system in a peer-to-peer (or distributed) network environment. The computer system 400 can also be implemented as or incorporated into various devices, such as a workstation that includes a controller, a stationary computer, a mobile computer, a personal computer (PC), a laptop computer, a tablet computer, or any other machine capable of executing a set of software instructions (sequential or otherwise) that specify actions to be taken by that machine. The computer system 400 can be incorporated as or in a device that in turn is in an integrated system that includes additional devices. In an embodiment, the computer system 400 can be implemented using electronic devices that provide voice, video or data communication. Further, while the computer system 400 is illustrated in the singular, the term “system” shall also be taken to include any collection of systems or sub-systems that individually or jointly execute a set, or multiple sets, of software instructions to perform one or more computer functions.

As illustrated in FIG. 4, the computer system 400 includes a processor 410. The processor 410 may be considered a representative example of a processor of a controller and executes instructions to implement some or all aspects of methods and processes described herein. The processor 410 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 410 is an article of manufacture and/or a machine component. The processor 410 is configured to execute software instructions to perform functions as described in the various embodiments herein. The processor 410 may be a general-purpose processor or may be part of an application specific integrated circuit (ASIC). The processor 410 may also be a microprocessor, a microcomputer, a processor chip, a controller, a microcontroller, a digital signal processor (DSP), a state machine, or a programmable logic device. The processor 410 may also be a logical circuit, including a programmable gate array (PGA), such as a field programmable gate array (FPGA), or another type of circuit that includes discrete gate and/or transistor logic. The processor 410 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 400 further includes a main memory 420 and a static memory 430, where memories in the computer system 400 communicate with each other and the processor 410 via a bus 408. Either or both of the main memory 420 and the static memory 430 may be considered representative examples of a memory of a controller, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 420 and the static memory 430 are articles of manufacture and/or machine components. The main memory 420 and the static memory 430 are computer-readable mediums from which data and executable software instructions can be read by a computer (e.g., the processor 410). Each of the main memory 420 and the static memory 430 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 400 further includes a video display unit 450, such as a liquid crystal display (LCD), an organic light emitting diode (OLED), a flat panel display, a solid-state display, or a cathode ray tube (CRT), for example. Additionally, the computer system 400 includes an input device 460, such as a keyboard/virtual keyboard or touch-sensitive input screen or speech input with speech recognition, and a cursor control device 470, such as a mouse or touch-sensitive input screen or pad. The computer system 400 also optionally includes a disk drive unit 480, a signal generation device 490, such as a speaker or remote control, and/or a network interface device 440.

In an embodiment, as depicted in FIG. 4, the disk drive unit 480 includes a computer-readable medium 482 in which one or more sets of software instructions 484 (software) are embedded. The sets of software instructions 484 are read from the computer-readable medium 482 to be executed by the processor 410. Further, the software instructions 484, when executed by the processor 410, perform one or more steps of the methods and processes as described herein. In an embodiment, the software instructions 484 reside all or in part within the main memory 420, the static memory 430 and/or the processor 410 during execution by the computer system 400. Further, the computer-readable medium 482 may include software instructions 484 or receive and execute software instructions 484 responsive to a propagated signal, so that a device connected to a network 401 communicates voice, video or data over the network 401. The software instructions 484 may be transmitted or received over the network 401 via the network interface device 440.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, fluid responsiveness monitoring enables ranking of clinically validated tests/procedures of fluid responsiveness based on automatically gathered patient information. The fluid responsiveness monitoring system and method described above can use the most preferred test and whenever possible conduct the test continuously and autonomously or when necessary, and guide the medical personnel through alternative tests that require medical personnel intervention. Thereby the fluid responsiveness monitoring system and method can support the medical personnel to optimize fluid therapy in critically ill patients by providing continuous reliable information of a patients' fluid responsiveness, ultimately improving patient outcomes.

Although fluid responsiveness monitoring has been described with reference to several exemplary embodiments, it is understood that the words that have been used are words of description and illustration, rather than words of limitation. Changes may be made within the purview of the appended claims, as presently stated and as amended, without departing from the scope and spirit of fluid responsiveness monitoring in its aspects. Although fluid responsiveness monitoring has been described with reference to particular means, materials and embodiments, fluid responsiveness monitoring is not intended to be limited to the particulars disclosed; rather fluid responsiveness monitoring extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72 (b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

We claim:

1. A system, comprising:
a controller with a memory that stores instructions, and a processor that executes the instructions, wherein, when executed by the processor, the instructions cause the system to:
automatically gather patient information of a patient;
rank clinically validated tests for fluid responsiveness based on the automatically gathered patient information;
identify a clinically validated test ranked highest;
repeatedly perform the clinically validated test ranked highest to monitor fluid responsiveness of the patient;
determine whether to continue performing the clinically validated test ranked highest; and
output guidance for performing an alternative clinically validated test based on determining not to continue performing the clinically validated test ranked highest.

2. The system of claim 1, wherein the clinically validated test ranked highest is performed autonomously.

3. The system of claim 1, further comprising:
a patient monitor,
wherein the instructions cause the system further to:
obtain patient data from the patient monitor; and
rank the clinically validated tests for fluid responsiveness based on the patient data from the patient monitor.

4. The system of claim 1, further comprising:
a ventilator,
wherein the instructions cause the system further to:
obtain ventilator data from the ventilator; and
rank the clinically validated tests for fluid responsiveness based on the ventilator data from the ventilator.

5. The system of claim 4, wherein the instructions further cause the system to:
obtain ventilator waveform data from the ventilator;
obtain hemodynamic waveform data; and
monitor fluid responsiveness of the patient based on the ventilator waveform data and the hemodynamic waveform data.

6. The system of claim 1, further comprising:
a display, wherein the instructions further cause the system to display a result of monitoring fluid responsiveness on the display.

7. The system of claim 6, wherein the instructions further cause the system to:
assess fluid responsiveness of the patient; and
when the fluid responsiveness of the patient is assessed to be uncertain, output the guidance for performing an alternative clinically validated test based on determining not to continue performing the clinically validated test ranked highest.

8. The system of claim 6, wherein the instructions further cause the system to:
generate a display on the display of at least one of patient fluid responsiveness, guidance for a manual test step, or guidance to change a protocol.

9. The system of claim 1, wherein the system ranks the clinically validated tests for fluid responsiveness based on the automatically gathered patient information by comparing prerequisites for the clinically validated tests with conditions of the patient.

10. A method for monitoring fluid responsiveness, comprising:
automatically gathering patient information of a patient;
ranking clinically validated tests for fluid responsiveness based on the automatically gathered patient information;
identifying a clinically validated test ranked highest;
repeatedly performing the clinically validated test ranked highest to monitor fluid responsiveness of the patient;
determining whether to continue performing the clinically validated test ranked highest; and
outputting guidance for performing an alternative clinically validated test based on determining not to continue performing the clinically validated test ranked highest.

11. The method of claim 10, wherein the clinically validated test ranked highest is performed autonomously.

12. The method of claim 10, further comprising:
obtaining patient data from a patient monitor; and
ranking the clinically validated tests for fluid responsiveness based on the patient data from the patient monitor.

13. The method of claim 10, further comprising:
obtaining ventilator data from a ventilator; and
ranking the clinically validated tests for fluid responsiveness based on the ventilator data from the ventilator.

14. The method of claim 13, further comprising:
obtaining ventilator waveform data from the ventilator;
obtaining hemodynamic waveform data; and
monitoring fluid responsiveness of the patient based on the ventilator waveform data and the hemodynamic waveform data.

15. The method of claim 14, further comprising:
displaying a result of monitoring fluid responsiveness on the display;
assessing fluid responsiveness of the patient; and
when the fluid responsiveness of the patient is assessed to be uncertain, outputting the guidance for performing an alternative clinically validated test based on determining not to continue performing the clinically validated test ranked highest.

16. The method of claim 14, further comprising:
generating a user interface on the display of at least one of patient fluid responsiveness, guidance for a manual test step, or guidance to change a protocol.

17. A tangible, non-transitory computer-readable medium that stores instructions, which when executed by a processor, cause the processor to:
automatically gather patient information of a patient;
rank clinically validated tests for fluid responsiveness based on the automatically gathered patient information;
identify a clinically validated test ranked highest;
repeatedly perform the clinically validated test ranked highest to monitor fluid responsiveness of the patient;
determine whether to continue performing the clinically validated test ranked highest; and
output guidance for performing an alternative clinically validated test based on determining not to continue performing the clinically validated test ranked highest.
